(19) United States
(12) Patent Application Publication
(10) Pub. No.: US 2010/0280519 A1
(43) Pub. Date: Nov. 4, 2010

(54) IMPLANTABLE DISTRACTOR WHOSE LENGTH CAN BE MODIFIED WITHOUT REOPERATION AND WHICH HAS A J-SHAPE

(76) Inventor: Arnaud Soubeiran, Paris (FR)

Correspondence Address: FOLEY HOAG, LLP;PATENT GROUP, WORLD TRADE CENTER WEST, 155 SEAPORT BLVD, BOSTON, MA, 02110, US

(21) Appl. No.: 12/601,723

(22) PCT Filed: May 25, 2008
(86) PCT No.: PCT/FR08/00721

§ 371 Date: May 20, 2010

(30) Foreign Application Priority Data
May 28, 2007 (FR) 0703752

(51) Int. Cl. A61B 17/60

(52) U.S. Cl. 606/90

(57) ABSTRACT

This distraction device comprises a rod (1), a second rod (2) and means to control the translation of one rod relative to the other (3), so as to cause the desired distraction or compression. Each rod (1) and (2) has a first end, respectively (11) and (21), which receive means to link to a part of the organism such as hooks or screws for vertebra, ribs or the pelvis, and a second end, respectively (12) and (22), linked to the means to control the translation of one rod relative to the other (3) such that the direction of the first end (11) toward the second end (12) of the first rod (1) is roughly the same as the direction of the first end (21) toward the second end (22) of the second rod (2). The distraction device thus roughly forms a J, the bulging and rigid part of which is located outside the area concerned by the surgical treatment, facilitating its implantation. It is useful for straightening of the spine and bone lengthening, deformity correction or transport, depending on the means used to link to the organism.

### TECHNICAL FIELD

The present invention concerns the field of implantable distraction devices, the length of which can be modified without reoperating, and in particular those used for surgery of the spine which are attached to one or more vertebra and/or one or more ribs and/or the pelvis.

### PRIOR ART

Such distraction devices are described in documents WO 08/040880 and WO 01/78614. In these documents the distraction devices comprise a first rod, a second rod and means to control the translation of one rod relative to the other so as to produce the desired distraction or compression. Each of the two said first and second rods comprises a first end able to receive the means to link to a part of the organism to which it is desired to attach the said rod, such as hooks or screws for the vertebra, ribs or pelvis, and a second end linked to the said means to control the translation of one rod relative to the other. However, in all these implantable distraction devices for which the length can be modified without reoperating, the direction of the first end toward the second end of the said first rod is substantially opposite the direction of the first end toward the second end of the said second rod. The said means to control the translation of one rod relative to the other is thus positioned in the area affected by the surgical treatment, for example between the vertebra or ribs to which the device is attached. This area is frequently very curved and sometimes short, as is often the case when the distraction device is used between ribs, and consequently implantation of this type of distraction device fitted with means to control the translation of one rod relative to the other, the rods being nondeformable, is often difficult or even impossible.

### SUMMARY OF THE INVENTION

The invention provides a solution to this problem. Indeed, the distraction device according to the invention comprises a first rod, a second rod, and means to control the translation of one rod relative to the other so as to produce the desired distraction or compression, wherein the said first rod comprises a first end able to receive the means to link to a part of the organism such as hooks or screws for vertebra, ribs or the pelvis, and a second end linked to the said means in order to control the translation of one rod relative to the other, and wherein the said second rod comprises a first end able to receive means to link to a part of the organism such as hooks or screws for vertebra, ribs or the pelvis, and a second end linked to the said means in order to control the translation of one rod relative to the other, and wherein both the said first and second rods are arranged such that the direction of the first end toward the second end of the said first rod is substantially the same as the direction of the first end toward the second end of the said second rod.

The said distraction device according to the invention substantially forms a “J” shape. The said means to control the translation of one rod relative to the other are positioned beneath the said “J”, outside the area concerned by the surgical treatment.

As a consequence, the bulging and rigid part, the shape of which cannot be changed during surgery, constituted by the said means to control the translation of one rod relative to the other, is located outside the area concerned by the surgical treatment, which substantially facilitates the implantation of the said distraction device.

In addition to surgery of the spine, this J-shaped distraction device structure can also be useful, in combination with appropriate means to link to the organism, gradually to separate two parts of a bone, in which an osteotomy has been made, either to lengthen it by bone distraction, or to reconstruct a missing part by bone transport, or again gradually to correct a deformation, as in the case of a tibial valgisation osteotomy, for example. In all these cases the structure of the invention enables means to be positioned to control the translation of one rod relative to the other at some distance from the osteotomy, in a more spacious area with better muscular coverage.

The invention can also be useful to distract soft tissues such as a retracted muscle, for example, such as the shoulder rotator. The J shape facilitates implantation and enables the elements which may interfere with medical imaging, such as an MRI machine, to be separated; these may be contained in the said means to control the translation of one rod relative to the other of the area to be treated in which the said imaging is required.

### BRIEF DESCRIPTION OF ILLUSTRATIONS

A better understanding of the invention will be obtained in the course of the following description on sight of the drawings appended for illustrative purposes, but not restrictively, in which

FIG. 1 represents a known distraction device as described in document WO 08/040880.

FIG. 2 represents a first preferred embodiment of the distraction device according to the invention using the means to control the translation of one rod relative to the other described in document WO 08/040880.

FIG. 3 represents a second preferred embodiment of the distraction device according to the invention also using the means to control the translation of one rod relative to the other described in document WO 08/040880.

It is clear that, in the figures, the same references designate the same elements, in whichever figure they appear and whatever the form of representation of these elements. Similarly, if elements are not specifically referenced in one of the figures the reference can easily be found by referring to another figure. In all the figures the dimensions and proportions have been altered when this was able to facilitate understanding. In all the figures only the parts required for understanding have been represented using dotted lines when they are not normally visible in the view.

### DESCRIPTION OF ILLUSTRATIVE EMBODIMENTS

The distraction devices represented in FIGS. 1 to 3 all comprise a first rod (1), a second rod (2) and the same means to control the translation of one rod relative to the other (3), so as to cause the desired distraction or compression. The said first rod (1) comprises a first end (11) able to receive the means to link to a part of the organism (not represented), for example such as hooks or screws for the vertebrae, ribs or pelvis, to which it is desired to attach it, and a second end (12) linked to the said means to control the translation of one rod relative to the other (3). The said second rod (2) comprises a first end (21) able to receive the means to link to a part of the organism, which are not represented, for example such as hooks or screws for the vertebrae, ribs or pelvis, to which it is desired to attach it, and a second end (22) linked to the said means to control the translation of one rod relative to the other (3).

The direction of a rod is defined as the direction of the first end toward the second end of the said rod.

In the known distraction device represented in FIG. 1, the said second rod (2) is assembled, aligned with, and in the opposite direction to, that of the first rod (1), on the said means to control the translation of one rod relative to the other.

In the preferred embodiments of the distraction device according to the invention represented respectively in FIG. 2 and in FIG. 3, the said second rod (2) is assembled on the said means to control the translation of one rod relative to the other (3) parallel to the said first rod (1), and in the same direction as it.

In the first preferred embodiment represented in FIG. 2 the said second rod (2) is linked to the said means to control the translation of one rod relative to the other (3) on the side on which the first rod (1) emerges. The said means to control the translation of one rod relative to the other (3) can thus be implanted beneath or above the area concerned by the surgical treatment.

In the second preferred embodiment represented in FIG. 3 the said second rod (2) is linked to the said means to control the translation of one rod relative to the other (3) on the side opposite to that on which the first rod (1) emerges. The said means to control the translation of one rod relative to the other (3) can thus be implanted parallel to the means to link to a part of the organism positioned on the said second rod (2).

Other embodiments of the invention are, naturally, possible, in particular with other means to control the translation of one rod relative to the other (3) in addition to those represented in FIGS. 1 to 3, for example those described in document WO 01/78614, and other positions and means to link the rods to the said means to control the translation of one rod relative to the other (3), or again other means to link to other parts of the organism, for example bones or even muscles, known to one of ordinary skill in the art, which also satisfy the definition of the invention. In the same way, embodiments of the invention in which the rods are roughly cylindrical have been presented, but there are other embodiments which are not outside the scope of the invention, in which the rods can have a square or rectangular section, flattened at one end, or again can have piercings or fluting, for example.

### POSSIBILITIES FOR INDUSTRIAL APPLICATION

The present invention is particularly useful for the production of all types of implantable distraction or compression device the length of which can be modified without reoperating for surgery of the spine, attached to one or more vertebrae and/or one or more ribs and/or to the pelvis. It can also be used in combination with appropriate means to link to the organism, in order gradually to separate two parts of a long bone, or such as the jaw, in which an osteotomy has been made, either to lengthen by bone distraction, or to reconstruct a missing portion by bone transport, or again gradually to correct a deformation.

1. An implantable distraction device the length of which can be modified without reoperating, comprising a first rod (1), a second rod (2), and means to control the translation of one rod relative to the other (3) so as to produce the desired distraction or compression, wherein the said first rod (1) comprises a first end (11) able to receive the means to link to a part of the organism such as hooks or screws for vertebrae, ribs or the pelvis, and a second end (12) linked to the said means in order to control the translation of one rod relative to the other (3), and the said second rod comprising a first end (21) able to receive means to link to a part of the organism such as hooks or screws for vertebrae, ribs or the pelvis, and a second end (22) linked to the said means in order to control the translation of one rod relative to the other (3), characterised in that the direction of the first end (11) toward the second end (12) of the said first rod (1) is substantially the same as the direction of the first end (21) toward the second end (22) of the said second rod (2).
